(19) United States
(12) Patent Application Publication
(10) Pub. No.: US 2010/0249112 A1
(43) Pub. Date: Sep. 30, 2010

(54) COMBINATION OF CHK AND PARP INHIBITORS FOR THE TREATMENT OF CANCERS

(75) Inventors: Mark James O'Connor, Cambridgeshire (GB); Graeme Cameron Murray Smith, Cambridgeshire (GB); Sonya Zabludoff, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA R&D, Sodertalje (SE)

(21) Appl. No.: 12/601,328

(22) PCT Filed: May 23, 2008
(86) PCT No.: PCT/GB2008/050372

§ 371 Date: May 20, 2010

Related U.S. Application Data
(60) Provisional application No. 60/940,203, filed on May 25, 2007.

(51) Int. Cl. A61K 31/551; A61K 31/502; A61K 31/5377; A61P 35/00

(52) U.S. Cl. 514/218; 514/248; 514/232.8

(57) ABSTRACT

A combination, comprising a checkpoint kinase (CHK) inhibitor, or a pharmaceutically acceptable salt thereof, and a poly (ADP-ribose)polymerase (PARP) inhibitor, or a pharmaceutically acceptable salt thereof is described.

### The present invention discloses therapies for treating cancer.

### BACKGROUND OF THE INVENTION

Chemotherapy and radiation exposure are currently the major options for the treatment of cancer, but the therapeutic utility of both these approaches is severely limited by drastic adverse effects on normal tissue, and the frequent development of tumor cell resistance. It is therefore highly desirable to improve the efficacy of cancer treatments in a way that does not increase the toxicity associated with them. In some cases, one way to achieve enhanced efficacy is by employing anticancer agents in combination, wherein said combination causes a better therapeutic effect than that seen with each drug alone.

Combined treatment regimens would add to the therapies available to patients suffering from cancer. For example, in one possible scenario, a drug may act to increase the sensitivity of the malignant cell to the other drug of a combination therapy. In other scenarios, combinations of anticancer agents may have additive, or even synergistic, therapeutic effects.

One particular class of therapeutic agent being tested in clinical trials for cancer are inhibitors of the mammalian enzyme poly (ADP-ribose)polymerase-1, also known as poly(ADP-ribose)synthase and poly ADP-ribosyltransferase, and commonly referred to as PARP-1. PARP-1 is the founding member of a family of 18 related enzymes. PARP-1 has been implicated in the signalling of DNA damage through its ability to recognize and rapidly bind to DNA single or double strand breaks (D'Amours et al, 1999, Biochem. J. 342: 249-268). Several observations have led to the conclusion that PARP participates in a variety of DNA-related functions including gene amplification, cell division, differentiation, apoptosis, DNA base excision repair and also effects on telomere length and chromosome stability. PARP-1 has also been associated with malignant transformation. For example, PARP activity is higher in the isolated nuclei of SV40-transformed fibroblasts, while both leukemic cells and colon cancer cells show higher enzyme activity than the equivalent normal leukocytes and colon mucosa (Miwa et al, 1977, Arch. Biochem. Biophys. 181: 313-321; Burzio et al, 1975, Proc. Soc. Exp. Biol. Med. 149: 933-938; and Hirai et al, 1983, Cancer Res. 43: 3441-3446). In preclinical models of cancer, PARP inhibitors have been shown to potentiate the effects of a wide range of chemotherapeutics and ionizing radiation. More recently, as a single agent, PARP inhibitors have been shown to be effective in the killing of cells defective in the DNA repair process of homologous recombination such as BRCA1 or BRCA2 mutant cells. Several PARP inhibitors have been described with some having entered clinical trials.

Another particular class of therapeutic agents that has the potential to treat cancer are inhibitors of the checkpoint kinase (CHK) such as checkpoint 1 kinase (CHK1). CHK1 is an important regulatory component of the cell cycle (See, for ex., Prudhomme, Recent Patents on Anti-Cancer Drug Discovery, 2006, 1:55). An individual cell replicates by making an exact copy of its chromosomes, and then segregating these into separate cells. This cycle of DNA replication, chromosome separation and division is regulated by mechanisms within the cell that maintain the order of the steps and ensure that each step is precisely carried out. Key to these processes are the cell cycle checkpoints (Hartwell et al., Science, Nov. 3, 1989, 246(4930):629-34) where cells may arrest to ensure DNA repair mechanisms have time to operate prior to continuing through the cycle into mitosis. Examples of checkpoints that are key in the regulation of the cell cycle are the G1/S checkpoint that is regulated by checkpoint kinase 2 (CHK2) and p53 and the intra-S and G2/M checkpoint that are monitored by the Ser/Thr kinase checkpoint kinase 1 (CHK1). As the cell cycle arrest induced by these checkpoints is a crucial mechanism by which cells can overcome the damage resulting from radio- or chemotherapy, their abrogation by novel agents should increase the sensitivity of tumor cells to DNA damaging therapies. One approach to the design of compounds that abrogate the G2/M checkpoint is to develop inhibitors of the key G2/M regulatory kinase CHK1, and this approach has been shown to work in a number of proof of concept studies. (Koniaras et al., Oncogene, 2001, 20:7453; Luo et al., Neoplasia, 2001, 3:411; Busby et al., Cancer Res., 2000, 60:2108; Jackson et al., Cancer Res., 2000, 60:566).

Several CHK inhibitors have been identified. These compounds include aminopyrazoles, indazoles, tricyclic compounds, ureas, carbamates, diazepinones, pyrimidines, benzimidazole quinolones and macrocyclic compounds. (See, e.g., Prudhomme, Recent Patents on Anti-Cancer Drug Discovery, 2006, 1:55 Janetka et al., Curr Opin Drug Discovery Dev 2007, 10(4)). 2-ureidothiophene compounds and 3-ureidothiophene compounds are described as CHK inhibitors in WO03029241 and WO03028731, respectively. In addition, fused triazolones are described as CHK inhibitors in WO2004/081008. CHK inhibitors also include the thiophene carboxamides disclosed in WO2005/016909; the thiophene carboxamides disclosed in WO 2005/066163; and the substituted heterocycles, described in WO2006/106326.

### SUMMARY OF THE INVENTION

The present invention relates to a combination comprising a checkpoint kinase (CHK) inhibitor, or a pharmaceutically acceptable salt thereof, and a poly (ADP-ribose) polymerase (PARP) inhibitor, or a pharmaceutically acceptable salt thereof. This combination has been found to be useful for its anti-proliferative (such as anti-cancer) activity and is therefore useful in methods of treatment of the human or animal body. The cancer can be in a metastatic state or a non-metastatic state. Examples of cancer include oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer, non small cell lung cancer (NSCLC), and small cell lung cancer (SCLC), gastric cancer, head and neck cancer, brain cancer, renal cancer, thyroid, lymphoma and leukaemia.

### BRIEF DESCRIPTION OF THE DRAWINGS

FIG. 1 shows an IC₅₀ plot of the combination of a CHK inhibitor and a PARP inhibitor with simultaneous addition and exposure in an NCI-H460 dominant negative (dn) p53 cell line.

FIG. 2 shows an IC₅₀ plot of the combination of a CHK inhibitor and a PARP inhibitor with simultaneous addition and exposure in NCI-H460dnp53 cell line

FIG. 3 shows an IC₅₀ plot of the combination of a PARP inhibitor and a CHK inhibitor with simultaneous addition and exposure in NCI-H460dnp53 cell line.

FIG. 4 shows an IC₅₀ plot of the combination of a CHK inhibitor followed by a PARP inhibitor in NCI-H460dnp53 cell line.

FIG. 5 shows an an IC₅₀ plot of the combination of a PARP inhibitor followed by CHK inhibitor in NCI-H460dnp53 cell line

FIG. 6 shows an IC₅₀ plot of the combination of CHK and a PARP inhibitor with simultaneous addition and exposure in SW620 cell line

### DETAILED DESCRIPTION OF THE INVENTION

The present invention relates to a combination comprising a CHK inhibitor, or a pharmaceutically acceptable salt thereof, and a PARP inhibitor, or a pharmaceutically acceptable salt thereof. This combination is useful for the treatment or prophylaxis of cancer.

### CHK Inhibitors

A “CHK inhibitor” refers to any compound or substance that can inhibit the activity of checkpoint 1 kinase (CHK) and/or the activity of checkpoint 2 kinase (CHK2). CHK inhibitors are known in the art and include aminopyrazoles, indazoles, tricyclic compounds, ureas, carbamates, diazepinones, pyrimidines, benzimidazole quinolones and macrocyclic compounds. It is understood that the CHK inhibitors for use in the methods of the present invention include compounds in free form or in the form of a pharmaceutically acceptable salt of the compound or in the form of a pharmaceutically acceptable solvate of the compound or salt. In particular, CHK inhibitors include the thiophene carboxamides disclosed in WO2005/066163 (These CHK inhibitors inhibit the activity of CHK1 and CHK2). These CHK inhibitors can be prepared in a number of ways well known to one skilled in the art of organic synthesis, including, but not limited to, the methods of synthesis described in detail in WO 2005/066163, the entire contents of which are hereby incorporated by reference. Thiophene carboxamides of interest as CHK inhibitors include compounds of the aforementioned WO 2005/066163 as shown in Formula (I):

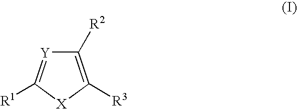

wherein:
X is selected from NH, S and O;
Y is selected from CH or N;
R¹ is selected from cyano, isocyano, C₁₋₆alkyl, —NR¹¹⁻¹², C₁₋₆alkoxy, C₂₋₆alkenyl, C₂₋₆alkynyl, cycloalkyl, cycloalkenyl, aryl, and heterocyclyl, provided R¹ is not thienyl; and wherein R¹ may be optionally substituted on one or more carbon atoms by one or more R⁹; and wherein if said R¹ contains an —NH— moiety, the nitrogen of said moiety may be optionally substituted by a group selected from R¹⁰;
R² and R³ are each independently selected from —C(═O)NR⁶R⁷, —SO₂NR¹⁶R¹⁷, —NHC(═O)NHR⁴, and —NHC(═NR⁸)NH₂;
R⁴ is selected from H, OH, —NR¹¹R¹², benzyl, C₁₋₆alkoxy, cycloalkyl, cycloalkenyl, aryl, heterocyclyl, mercapto, CHO, —COaryl, —CO(C₁₋₆alkyl), —CONR³⁰R³¹, —CO₂(C₁₋₆alkyl), —CO₂aryl, —CO₂NR³⁰R³¹, —Salkyl, —SO(C₁₋₆alkyl), —SO₂(C₁₋₆alkyl), —Saryl, —SOaryl, —SO₂aryl, —SO₂NR³⁰R³¹, and —(C₁₋₆alkyl)SO₂ NR³⁰R³¹ wherein R⁴ may be optionally substituted on one or more carbon atoms by one or more R¹⁵; and wherein if said heterocyclyl contains a —NH- moiety, the nitrogen may be optionally substituted by a group selected from R¹⁴;
R⁶ and R⁷ are each independently selected from H, OH, OCH₃, C₁₋₆alkoxy, —NH₂, —NHCH₃, —N(CH₃)₂, (C₁₋₃alkyl)NR¹¹R¹², —CH₂CH₂OH, cycloalkyl, and a 5, 6, or 7-membered heterocyclyl ring containing at least one nitrogen atom, provided R⁶ and R⁷ are not both H; alternatively R⁶ and R⁷ taken together with the N to which they are attached form a heterocyclic ring; wherein R⁶ and R⁷ independently of each other may be optionally substituted on one or more carbon atoms by one or more R¹⁸; and wherein if said heterocyclyl contains a —NH—moiety, the nitrogen of said moiety may be optionally substituted by a group selected from R¹⁹;
R⁸ is selected from cyano, isocyano, —SO₂(C₁₋₆alkyl), —SO₂-aryl; —SO₂cycloalkyl, —SO₂cycloalkenyl, —SO₂heterocyclyl, and CF₃; wherein R⁸ may be optionally substituted on one or more carbon atoms by one or more R²³;
R⁹, R¹⁵, R¹⁸, R²³, R²⁴ and R³³ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R⁹, R¹⁵, R¹⁸,R²³, R²⁴ and R³³ independently of each other may be optionally substituted on carbon by one or more R²⁰ and on nitrogen of any moiety that contains an NH or NH₂ by R²¹;

R¹⁰, R¹⁴, R¹⁹, R²⁵ and R³⁴ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R¹⁰,R¹⁴,R¹⁹, R²⁵ and R³⁴ independently of each other may be optionally substituted on carbon by one or more R²² and on nitrogen of any moiety that contains an NH or NH₂ by R²³ ;
R¹¹ and R¹² are independently selected from H, C₁₋₆alkyl, cycloalkyl, aryl, heterocyclyl; alternatively R¹¹ and R¹² taken together with the N to which they are attached form a heterocyclic ring; wherein R¹¹ and R¹² independently of each other may be optionally substituted on carbon by one or more R³³, and wherein if said heterocyclyl contains a —NH— moiety, the nitrogen of said moiety may be optionally substituted by a group selected from R³⁴;
R¹⁶ and R¹⁷ are each independently selected from H, OH, OCH₃, C₁₋₆alkoxy, NH₂, —NHCH₃, —N(CH₃)₂, (C₁₋₃alkyl)NR¹¹R¹², —CH₂CH₂OH, cycloalkyl, aryl, or a 5, 6 or 7-membered heterocyclyl ring containing at least one nitrogen atom, provided R¹⁶ and R¹⁷ are not both H; alternatively R¹⁶ and R¹⁷ taken together with the N to which they are attached form an optionally substituted heterocyclic ring; wherein R¹⁶ and R¹⁷ independently of each other may be optionally substituted on one or more carbon atoms by one or more R²⁴; and wherein if said heterocyclyl contains an —NH— moiety, the nitrogen of said moiety may be optionally substituted by a group selected from R²⁵;
R²⁰, R²² and R³² are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —SO(C₁₋₆alkyl), —SO₂(C₁₋₆alkyl), —SO₂NR³⁰R³¹; wherein R²⁰, R²¹ and R³² independently of each other may be optionally substituted on carbon by one or more R²⁶ and on nitrogen of any moiety that contains an NH or NH₂ by R²⁷;
R²¹, R²³ and R³⁵ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆ alkyl), —COheterocyclyl, —COcycloalkyl, —CO₂H, —CO₂(C₁₋₆ alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R²¹, R²³ and R³⁵ independently of each other may be optionally substituted on carbon by one or more R²⁸ and on nitrogen of any moiety that contains an NH by R²⁹;
R²⁶ and R²⁸ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R²⁷ and R²⁹ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R³⁰ and R³¹ are each independently selected from halogen, nitro, —NH₂, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR¹¹R¹², —N(C₁₋₆alkyl)CONR¹¹R¹², —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO2NR¹¹R¹²; wherein R³⁰ and R³¹ independently of each other may be optionally substituted on carbon by one or more R³²; and wherein if said heterocyclyl contains a —NH—or NH₂ moiety, the nitrogen of said moiety may be optionally substituted by a group selected from R³⁵; or a pharmaceutically acceptable salt thereof;
provided that when X is S; Y is CH; R₂ is C(═O)NR⁶R⁷; and R³ is NHC(═O)NHR⁴; then R¹ cannot be

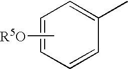

wherein R⁵ is selected from H, optionally substituted carbocyclyl, or optionally substituted
C₁₋₆alkyl; with the further proviso that said compound is not
5-Methyl-2-ureido-thiophene-3-carboxylic acid (1-ethyl-piperidin-3-yl)-amide;
[3-((S)-3-Amino-azepane-1-carbonyl)-5-ethyl-thiophen-2-yl]-urea;

2-Morpholin-4-yl-4-ureido-thiazole-5-carboxylic acid (S)-piperidin-3-ylamide;
2-Methyl-5-ureido-oxazole-4-carboxylic acid (S)-piperidin-3-ylamide;
5-(4-CHloro-phenyl)-3-{3-[(R)-1-(2,2,2-trifluoro-acetyl)-piperidin-3-yl]-ureido}-thiophene-2-carboxylic acid (S)-piperidin-3-ylamide; or
N-(3-{[(3S)-3-aminoazepan-1-yl]carbonyl}-5-pyridin-2-yl-2-thienyl)urea.

Compounds of Formula (I) which are of particular interest include the following:
5-(3-Fluoro-phenyl)-3-ureido-thiophene-2-carboxylic acid (S)-piperidin-3-ylamide;
5-Phenyl-2-ureido-thiophene-3-carboxylic acid (S)-piperidin-3-ylamide;
5-(3,5-Difluoro-phenyl)-2-ureido-thiophene-3-carboxylic acid (S)-piperidin-3-ylamide;
5-(4-Fluoro-phenyl)-2-ureido-thiophene-3-carboxylic acid (S)-piperidin-3-ylamide;
5-(4-CHloro-phenyl)-2-ureido-thiophene-3-carboxylic acid (S)-piperidin-3-ylamide;
5-(3-CHloro-phenyl)-2-ureido-thiophene-3-carboxylic acid (S)-piperidin-3-ylamide;
5-[4-(Piperidine-1-carbonyl)-phenyl]-2-ureido-thiophene-3-carboxylic acid (S)-piperidin-3-ylamide;
5-(4-Cyano-phenyl)-3-ureido-thiophene-2-carboxylic acid (S)-piperidin-3-ylamide;
5-[4-(Piperidine-1-carbonyl)-phenyl]-3-ureido-thiophene-2-carboxylic acid (S)-piperidin-3-ylamide;
5-(3,4-Difluoro-phenyl)-3-ureido-thiophene-2-carboxylic acid (S)-piperidin-3-ylamide;
5-(3-CHloro-phenyl)-3-ureido-thiophene-2-carboxylic acid (S)-piperidin-3-ylamide;
5-(2,3-Difluoro-phenyl)-3-ureido-thiophene-2-carboxylic acid (S)-piperidin-3-ylamide;
5-(2,4-Difluoro-phenyl)-3-ureido-thiophene-2-carboxylic acid (S)-piperidin-3-ylamide;
5-(3,5-Difluoro-phenyl)-3-ureido-thiophene-2-carboxylic acid (S)-piperidin-3-ylamide;
5-Phenyl-3-ureido-thiophene-2-carboxylic acid (S)-piperidin-3-ylamide;
5-(4-CHloro-phenyl)-3-ureido-thiophene-2-carboxylic acid (S)-piperidin-3-ylamide.

Additional CHK inhibitors include the substituted heterocycles disclosed in WO2006/106326, which is incorporated by reference herein.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

Definitions for use with CHK inhibitors of Formula (I) above. As used in this application, the term “optionally substituted,” means that substitution is optional and therefore it is possible for the designated atom to be unsubstituted. In the event a substitution is desired then such substitution means that any number of hydrogens on the designated atom is replaced with a selection from the indicated group, provided that the normal valency of the designated atom is not exceeded, and that the substitution results in a stable compound. For example when a substituent is keto (i.e., ═O), then 2 hydrogens on the atom are replaced. When a group is indicated to be “optionally substituted” or “substituted” unless otherwise expressly stated examples of suitable substituents include the following: halogen, nitro, amino, cyano, trifluoromethyl, methyl, ethyl, alkyl, alkenyl, alkynyl, haloalkyl, alkoxy, hydroxy, alkylhydroxy, carbonyl, keto, —CH(OH)CH₃, —CH₂NH-alkyl-OH, alkyl-(OH)CH₃, —Oalkyl, —OCOalkyl, —NHCHO, —N-(alkyl)—CHO, —NH—CO-amino, —N-(alkyl)-CO-amino, —NH—COalkyl, —N-(alkyl)—COalkyl, -carboxy, -amidino, —CO-amino, —CO-alkyl, —CO₂alkyl, mercapto, —Salkyl, —SO(alkyl), —SO₂(alkyl), —SO₂-amino, -alkylsulfonylamino, phenyl, cycloalkyl, heterocyclic and heteroaryl, -alkly—NH-cycloalkyl, -alkyl—NH-optionally substituted heterocyclyl, -alkyl—NH-alkyl-OH, —C(═O)OC(CH₃)₃, —N(CH₃)₂, -alkyl-NH-alkyl-optionally substituted heterocyclyl, alkyl-aryl, alkyl-polycyclyl, alkyl-amino, alkyl-hydroxy, —CH₂NH-alkyl-heterocyclyl, —CH₂NHCH2CH(CH₃)₂ If the group to be substituted is a ring, the optional substituents could also be selected from:vicinal —O(alkyl)O—, vicinal —OC(haloalkyl)O—, vicinal —CH₂O(alkyl)O—, vicinal —S(alkyl)S— and —O(alkyl)S—. Each of these substituents can, themselves, be further substituted. Suitable examples of such further substitution include any of the foregoing suitable substituents.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms. Unless otherwise specified, “alkyl” general includes both saturated alkyl and unsaturated alkyl.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms. When cycloalkyl contains more than one ring, the rings may be fused or unfused and include bicyclo radicals. Fused rings generally refer to at least two rings sharing two atoms therebetween.

The term “cycloalkenyl” used alone or as suffix or prefix, refers to a monovalent ring-containing hydrocarbon radical having at least one carbon-carbon double bond and comprising at least 3 up to about 12 carbon atoms. When cycloalkenyl contains more than one ring, the rings may be fused or unfused and include bicyclo radicals.

The term “aryl” used alone or as suffix or prefix, refers to a hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring.

The term “alkoxy” used alone or as a suffix or prefix, refers to radicals of the general formula —O—R, wherein —R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “carbocyclyl” is intended to include both alicyclic and aromatic ring structures wherein the closed ring is made of carbon atoms. These may include fused or bridged polycyclic systems. Carbocyclyls may have from 3 to 10 carbon atoms in their ring structure, and often have 3, 4, 5, 6 and 7 carbon atoms in the ring structure. For example, “C₃₋₇ carbocyclyl” denotes such groups as cyclopropyl, cyclobutyl, cyclopentyl, cyclohexyl, cycloheptyl, cyclopentadiene or phenyl.

The term” or “heterocyclyl” used alone or as a suffix or prefix, refers to a ring-containing structure or molecule having one or more multivalent heteroatoms, independently 14 atoms in the ring(s). Heterocyclyl may be saturated or unsaturated, containing one or more double bonds, and heterocyclyl may contain more than one ring. When a heterocyclyl contains more than one ring, the rings may be fused or unfused. Fused rings generally refer to at least two rings sharing two atoms therebetween. Heterocyclyl may have aromatic character or may not have aromatic character.

Examples of heterocyclyls include, but are not limited to, 1H-indazolyl, 2-pyrrolidonyl, 2H, 6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azepanyl, azetidinyl, aziridinyl, azocinyl, benzimidazolyl, benzofuranyl, benzofuranyl, benzothiofuranyl, benzothiophenyl, benzodioxolyl, benzoxazolyl, benzthiophenyl, benzthiazolyl, benzotriazolyl, benzotetrazolyl, benzisoxazolyl, benzthiazole, benzisothiazolyl, benzimidazolyls, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dioxolanyl, furyl, 2,3-dihydrofuranyl, 2,5-dihydrofuranyl, dihydrofuro[2,3-b]tetrahydrofuranyl, furanyl, furazanyl, homopiperidinyl, imidazolyl, imidazolidinyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxiranyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, piperidinyl, pteridinyl, piperidonyl, 4-piperidonyl, purinyl, pyranyl, pyrrolidinyl, pyrrolinyl, pyrrolidinyl, pyrazinyl, pyrazolidinyl, pyrazolinyl, pyrazolyl, pyridazinyl, pyridooxazolyl, pyridoimidazolyl, pyridothiazolyl, pyridinyl, N-oxide-pyridinyl, pyridyl, pyrimidinyl, pyrrolidinyl, pyrrolinyl, pyrrolyl, pyridinyl, quinazolinyl, quinolinyl, 4H-quinolizinyl, quinoxalinyl, quinuclidinyl, carbolinyl, tetrahydrofuranyl, tetrahydroisoquinolinyl, thiophanyl, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiiranyl, triazinyl, 1,2,3-triazolyl, 1,2,4-triazolyl, 1,2,5-triazolyl, 1,3,4-triazolyl, and xanthenyl.

The terms “seven-membered”, “six-membered” and “five-membered” used as prefix refers to groups having a ring that contains, respectively, seven, six, and five ring atoms.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “amine” or “amino” used alone or as a suffix or prefix, refers to radicals of the general formula —NRR′, wherein R and R′ are independently selected from hydrogen or a hydrocarbon radical.

The term halogen includes fluorine, chlorine, bromine and iodine.

“Halogenated,” used as a prefix of a group, means one or more hydrogens on the group are replaced with one or more halogens.

“RT” or “rt” means room temperature.

When any variable (e.g., R¹, R⁴, R^{a}, R^{e} etc.) occurs more than one time in any constituent or formula for a compound, its definition at each occurrence is independent of its definition at every other occurrence. Thus, for example, if a group is shown to be substituted with 0-3 R¹, then said group may optionally be substituted with 0,1, 2 or 3 R¹ groups and R^{e} at each occurrence is selected independently from the definition of R^{e}. Also, combinations of substituents and/or variables are permissible only if such combinations result in stable compounds.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

When a circle is shown within a ring structure, i.e.

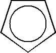

it indicates that the ring system is aryl or heteroaryl.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P.G.M. Protective Groups in Organic Synthesis, 3^{rd} ed.; Wiley: N.Y., 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Methods of making these compounds are known in the art as described in WO2005/066163, which is incorporated herein by reference.

PARP Inhibitors:

A “PARP inhibitor” refers to any agent that can inhibit the activity of PARP, for example, any one or more of PARP 1-18. Preferably the agent is a small molecule inhibitor. In one embodiment, the PARP inhibitor inhibits the activity of PARP1 and/or PARP2. PARP inhibitors are known in the art and include: Nicotinamides, such as 5-methyl nicotinamide and O-(2-hydroxy-3-piperidino-propyl)-3-carboxylic acid amidoxime, and analogues and derivatives thereof. Benzamides, including 3-substituted benzamides such as 3-aminobenzamide, 3-hydroxybenzamide 3-nitrosobenzamide, 3-methoxybenzamide and 3-chloroprocainamide, and 4-aminobenzamide, 1,5-di[(3-carbamoylphenyl)aminocarbonyloxy]pentane, and analogues and derivatives thereof. Isoquinolinones and Dihydroisoquinolinones, including 2H-isoquinolin-1-ones, 3H-quinazolin-4-ones, 5-substituted dihydroisoquinolinones such as 5-hydroxy dihydroisoquinolinone, 5-methyl dihydroisoquinolinone, and 5-hydroxy isoquinolinone, 5-amino isoquinolin-1-one, 5-dihydroxyisoquinolinone, 3,4 dihydroisoquinolin-1(2H)-ones such as 3,4 dihydro-5-methoxy-isoquinolin-1(2H)-one and 3, 4 dihydro-5-methyl-1(2H)isoquinolinone, isoquinolin-1(2H)-ones, 4,5-dihydro-imidazo[4,5,1-ij]quinolin-6-ones, 1,6,-naphthyridine-5(6H)-ones, 1,8-naphthalimides such as 4-amino-1,8-naphthalimide, isoquinolinone, 3,4-dihydro-5-[4-1(1-piperidinyl)butoxy]-1(2H)-isoquinolinone, 2,3-dihydrobenzo[de]isoquinolin-1-one, 1-11b-dihydro-[2H]benzopyrano[4,3,2-de]isoquinolin-3-one, and tetracyclic lactams, including benzpyranoisoquinolinones such as benzopyrano[4,3,2-de]isoquinolinone, and analogues and derivatives thereof. Benzimidazoles and indoles, including benzoxazole-4-carboxamides, benzimidazole-4-carboxamides, such as 2-substituted benzoxazole 4-carboxamides and 2-substituted benzimidazole 4-carboxamides such as 2-aryl benzimidazole 4-carboxamides and 2-cycloalkylbenzimidazole-4-carboxamides including 2-(4-hydroxphenyl)benzimidazole 4-carboxamide, quinoxalinecarboxamides, imidazopyridinecarboxamides, 2-phenylindoles, 2-substituted benzoxazoles, such as 2-phenyl benzoxazole and 2-(3-methoxyphenyl)benzoxazole, 2-substituted benzimidazoles, such as 2-phenyl benzimidazole and 2-(3-methoxyphenyl)benzimidazole, 1,3,4,5 tetrahydro-azepino[5,4,3-cd]indol-6-one, azepinoindoles and azepinoindolones such as 1,5 dihydro-azepino[4,5,6-cd]indolin-6-one and dihydrodiazapinoindolinone, 3-substituted dihydrodiazapinoindolinones such as 3-(4-trifluoromethylphenyl)-dihydrodiazapinoindolinone, tetrahydrodiazapinoindolinone and 5,6,-dihydroimidazo[4,5,1-j,k][1,4]benzodiazopin-7(4H)-one, 2-phenyl-5,6-dihydro-imidazo[4,5,1-jk][1,4]benzodiazepin-7(4H)-one and 2,3, dihydro-isoindol-1-one, and analogues and derivatives thereof. Phthalazin-1(2H)-ones and quinazolinones, such as 4-hydroxyquinazoline, phthalazinone, 5-methoxy-4-methyl-1(2) phthalazinones, 4-substituted phthalazinones, 4-(1-piperazinyl)-1(2H)-phthalazinone, tetracyclic benzopyrano[4,3,2-de]phthalazinones and tetracyclic indeno[1,2,3-de]phthalazinones and 2-substituted quinazolines, such as 8-hydroxy-2-methylquinazolin-4-(3H) one, tricyclic phthalazinones and 2-aminophthalhydrazide, and analogues and derivatives thereof. Isoindolinones and analogues and derivatives thereof. Phenanthridines and phenanthridinones, such as 5[H]phenanthridin-6-one, substituted 5[H]phenanthridin-6-ones, especially 2-, 3-substituted 5[H]phenanthridin-6-ones and sulfonamide/carbamide derivatives of 6(5H)phenanthridinones, thieno[2,3-c]isoquinolones such as 9-amino thieno[2,3-c]isoquinolone and 9-hydroxythieno[2,3-c]isoquinolone, 9-methoxythieno[2,3-c]isoquinolone, and N-(6-oxo-5,6-dihydrophenanthridin-2-yl]-2-(N,N-dimethylamino}acetamide, substituted 4,9-dihydrocyclopenta[lmn]phenanthridine-5-ones, and analogues and derivatives thereof. Benzopyrones such as 1,2-benzopyrone 6-nitrosobenzopyrone, 6-nitroso 1,2-benzopyrone, and 5-iodo-6-aminobenzopyrone, and analogues and derivatives thereof. Unsaturated hydroximic acid derivatives such as O-(3-piperidino-2-hydroxy-1-propyl)nicotinic amidoxime, and analogues and derivatives thereof. Pyridazines, including fused pyridazines and analogues and derivatives thereof. Other compounds such as caffeine, theophylline, and thymidine, and analogues and derivatives thereof.

Additional PARP inhibitors are described for example in U.S. Pat. No. 060,229,351,U.S. Pat. No. 7,041,675, WO07041357, WO2003057699 U.S. Pat. No. 06,444,676; US20060229289; US20060063926; WO2006033006; WO2006033007; WO03051879; WO2004108723; WO2006066172; WO2006078503; US20070032489; WO2005023246; WO2005097750; WO2005123687; WO2005097750; U.S. Pat. No. 7087637; U.S. Pat. No. 6,903,101; WO20070011962; US 20070015814; WO2006135873; UA20070072912; WO2006065392; WO2005012305; WO2005012305; EP412848; EP453210; EP454831; EP879820; EP879820; WO030805; WO03007959; U.S. Pat. No. 6,989,388; US20060094746; EP1212328; WO2006078711; U.S. Pat. No. 0,6426,415; U.S. Pat. No. 06,514,983; EP1212328; US2004 0254372; US2005 0148575; US2006 0003987; U.S. Pat. No. 06,635,642; WO200116137; WO2004105700; WO03057145A2; WO2006078711; WO2002044157; US2005 6924284; WO2005112935; US2004 6828319; WO2005054201;WO2005054209; WO2005054210; WO2005058843; WO2006003146; WO2006003147; WO2006003148; WO2006 003150; WO2006003146; WO2006003147; UA20070072842; U.S. Pat. No. 05587384; US2006 0094743; WO2002094790; WO2004048339; EP1582520; US20060004028; WO2005108400;U.S. Pat. No. 6,964,960; WO20050080096; WO2006137510; UA20070072841; WO2004087713; WO2006046035; WO2006008119; WO06008118; WO2006042638; US20060229289; US20060229351; WO2005023800; WO1991007404; WO2000042025; WO2004096779; U.S. Pat. No. 06,426,415; WO02068407; U.S. Pat. No. 06,476,048; WO2001090077; WO2001085687; WO2001085686; WO2001079184; WO2001057038; WO2001023390; WO01021615A1WO2001016136; WO2001012199; WO95024379, WO200236576; WO2004080976, Banasik et al. J. Biol. Chem., 267:3, 1569-75 (1992), Banasik et al. Molec. Cell. Biochem. 138:185-97 (1994)), Cosi (2002) Expert Opin. Ther. Patents 12 (7), and Southan & Szabo (2003) Curr Med Chem 10 321-340 and references therein.

In one aspect of the invention, the PARP inhibitor can be selected from a compound of Formula (Ib):

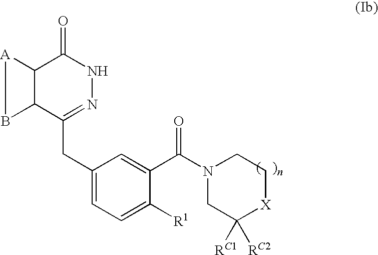

and isomers, salts, solvates, chemically protected forms, and prodrugs thereof wherein:
A and B together represent an optionally substituted, fused aromatic ring;
X can be NR^{X} or CR^{X}R^{Y};
if X═NR^{X} then n is 1 or 2 and if X═CR^{X}R^{Y} then n is 1;
R^{X} is selected from the group consisting of H, optionally substituted C₁₋₂₀ alkyl, C₅₋₂₀ aryl, C₃₋₂₀heterocyclyl, amido, thioamido, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are both hydrogen, or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo.

Therefore, if X is CR^{X}R^{Y}, then n is 1, the compound is of formula (Aa):

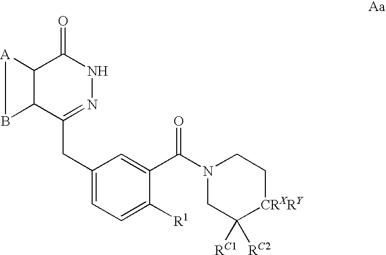

If X is NR^{X}, and n is 1. the compound is of formula (Ab):

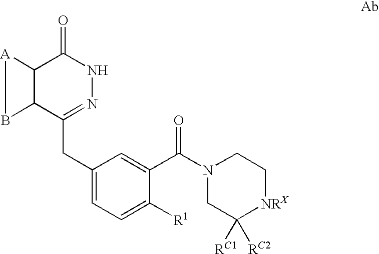

If X is NR^{X}, and n is 2, the compound is of formula (Ac):

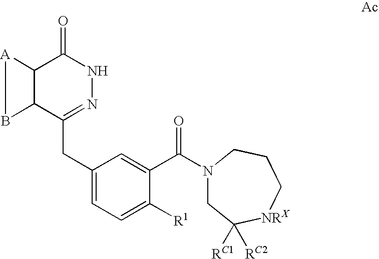

In one embodiment, the PARP inhibitor is selected from the following PARP inhibitors:

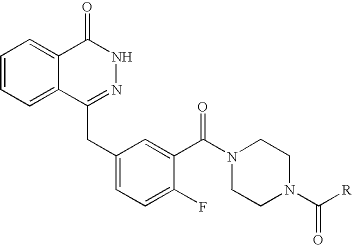

| Compound | R |
|---|---|
| 166 | 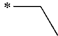 |
| 167 | 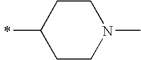 |
| 168 | 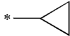 |
| 169 | 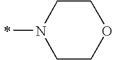 |
| 170 | 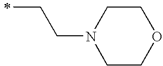 |
| 171 | 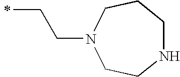 |
| 172 | 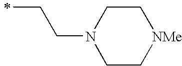 |
| 173 | 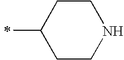 |

### Further Preferences

The following preferences can apply to each aspect of the PARP inhibitors, where applicable.

In the present invention, the fused aromatic ring(s) represented by -A-B- preferably consist of solely carbon ring atoms, and thus may be benzene, naphthalene, and is more preferably benzene. As described above, these rings may be substituted, but in some embodiments are preferably unsubstituted.

If the fused aromatic ring represented by -A-B- bears a substituent group, it is preferably attached to the atom which itself is attached to the central ring meta- to the carbonyl group. Thus, if the fused aromatic ring is a benzene ring, the preferred place of substitution is shown in the formula below by *:

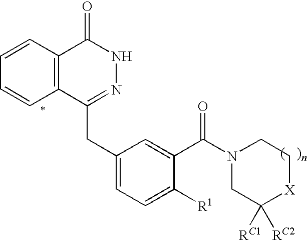

which is usually termed the 5-position of the phthalazinone moiety.

R^{l} is preferably selected from H, Cl and F, and is more preferably F.

It is preferred that R^{C1} and R^{C2} are both hydrogen.

When n is 2, X is NR^{X}. In these embodiments, R^{X} is preferably selected from the group consisting of: H; optionally substituted C₁₋₂₀ alkyl; optionally substituted C₅₋₂₀ aryl; optionally substituted ester groups, wherein the ester substituent is preferably C₁₋₂₀ alkyl; optionally substituted acyl groups; optionally substituted amido groups; optionally substituted thioamido groups; and optionally substituted sulfonyl groups. R^{X} is more preferably selected from the group consisting of: H; optionally substituted C₁₋₂₀ alkyl; optionally substituted C₅₋₂₀ aryl; and optionally substituted ester groups, wherein the ester substituent is preferably C₁₋₂₀ alkyl.

When n is 1, X may be NR^{X} or CR^{X}CR^{Y}.

In embodiments where X is NR^{X}, R^{X} is preferably selected from the group consisting of: H; optionally substituted C₁₋₂₀ alkyl; optionally substituted C₅₋₂₀ aryl; optionally substituted acyl; optionally substituted sulfonyl; optionally substituted amido; and optionally substituted thioamido groups.

In embodiments where X is CR^{X}R^{Y}, R^{Y} is preferably H. R^{X} is preferably selected from the group consisting of: H; optionally substituted C₁₋₂₀ alkyl; optionally substituted C₅₋₂₀ aryl; optionally substituted C₃₋₂₀ heterocyclyl; optionally substituted acyl, wherein the acyl substituent is preferably selected from C₅₋₂₀ aryl and C₃₋₂₀ heterocylyl (e.g. piperazinyl); optionally substituted amido, wherein the amino groups are preferably selected from H and C₁₋₂₀ alkyl or together with the nitrogen atom, form a C₅₋₂₀ heterocyclic group; and optionally substituted ester groups, wherein the ester substituent is preferably selected from C₁₋₂₀ alkyl groups.

Particularly preferred compounds include: 1, 2, 3, 4, 10, 21, 74, 97, 152, 153, 163, 167, 169, 173, 185, 232, 233, 250, 251, 252, 260 and 263.

Where appropriate, the above preferences may be taken in combination with each other.

Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms.

Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-CHlorophenyl. However, a reference to a class of structures may well include structurally isomeric forms falling within that class (e.g., C₁₋₇ alkyl includes n-propyl and iso-propyl; butyl includes n-, iso-, sec-, and tent-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Particularly relevant to the present invention is the tautomeric pair illustrated below:

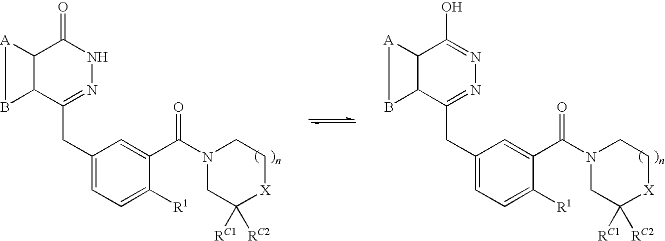

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below, as well as its different polymorphic forms.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl(diphenylmethyl), or trityl(triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (-OH), can be made into phosphonate prodrugs by reaction with chlorodibenzylphosphite, followed by hydrogenation, to form a phosphonate group —O—P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Methods of making these compounds are known in the art, for example, as described in WO2004/080976, which is incorporated herein by reference.

Definitions for use with PARP inhibitors of formula (Ib) above: The term “aromatic ring” is used herein in the conventional sense to refer to a cyclic aromatic structure, that is, a cyclic structure having delocalised 7c-electron orbitals.

The aromatic ring fused to the main core, i.e. that formed by -A-B-, may bear further fused aromatic rings (resulting in, e.g. naphthyl or anthracenyl groups). The aromatic ring(s) may comprise solely carbon atoms, or may comprise carbon atoms and one or more heteroatoms, including but not limited to, nitrogen, oxygen, and sulfur atoms. The aromatic ring(s) preferably have five or six ring atoms.

The aromatic ring(s) may optionally be substituted. If a substituent itself comprises an aryl group, this aryl group is not considered to be a part of the aryl group to which it is attached. For example, the group biphenyl is considered herein to be a phenyl group (an aryl group comprising a single aromatic ring) substituted with a phenyl group. Similarly, the group benzylphenyl is considered to be a phenyl group (an aryl group comprising a single aromatic ring) substituted with a benzyl group.

In one group of preferred embodiments, the aromatic group comprises a single aromatic ring, which has five or six ring atoms, which ring atoms are selected from carbon, nitrogen, oxygen, and sulfur, and which ring is optionally substituted. Examples of these groups include, but are not limited to, benzene, pyrazine, pyrrole, thiazole, isoxazole, and oxazole. 2-Pyrone can also be considered to be an aromatic ring, but is less preferred.

If the aromatic ring has six atoms, then preferably at least four, or even five or all, of the ring atoms are carbon. The other ring atoms are selected from nitrogen, oxygen and sulphur, with nitrogen and oxygen being preferred. Suitable groups include a ring with: no hetero atoms (benzene); one nitrogen ring atom (pyridine); two nitrogen ring atoms (pyrazine, pyrimidine and pyridazine); one oxygen ring atom (pyrone); and one oxygen and one nitrogen ring atom (oxazine).

If the aromatic ring has five ring atoms, then preferably at least three of the ring atoms are carbon. The remaining ring atoms are selected from nitrogen, oxygen and sulphur. Suitable rings include a ring with: one nitrogen ring atom (pyrrole); two nitrogen ring atoms (imidazole, pyrazole); one oxygen ring atom (furan); one sulphur ring atom (thiophene); one nitrogen and one sulphur ring atom (isothiazole, thiazole); and one nitrogen and one oxygen ring atom (isoxazole or oxazole).

The aromatic ring may bear one or more substituent groups at any available ring position. These substituents are selected from halo, nitro, hydroxy, ether, thiol, thioether, amino, C₁₋₇ alkyl, C₃₋₂₀ heterocyclyl and C₅₋₂₀ aryl. The aromatic ring may also bear one or more substituent groups which together form a ring. In particular these may be of formula —(CH₂)ₘ— or —O—(CH₂)ₚ—O—, where m is 2, 3, 4 or 5 and p is 1, 2 or 3.

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl(allyl, —CH—CH═CH₂), isopropenyl (1-methylvinyl, 13 C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —CCH) and 2-propynyl (propargyl, —CH₂—CCH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from:
saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);
unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);
saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);
unsaturated polycyclic hydrocarbon compounds: camphene (C₁₀), limonene (C₁₀), pinene (C₁ₒ);
polycyclic hydrocarbon compounds having an aromatic ring:

indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydropyran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆); N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring joined to another ring by a single atom common to both rings.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan(oxole), thiophene(thiole), pyrrole(azole), imidazole(1,3-diazole), pyrazole(1,2-diazole), triazole, oxazole, isoxazole, thiazole, isothiazole, oxadiazole, tetrazole and oxatriazole; and C₆ heteroaryl groups derived from isoxazine, pyridine(azine), pyridazine (1,2-diazine), pyrimidine(1,3-diazine; e.g., cytosine, thymine, uracil), pyrazine(1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Phbenzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cylic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

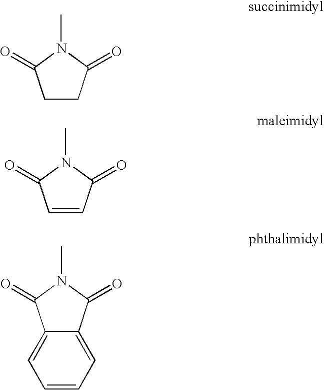

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R^{l} and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

CHK and PARP Combination

The term “combination” refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the benefit of the synergistic and /or additive effect of the combination.

Treatment

Where cancer is referred to, it can refer to the treatment of oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, brain cancer such as glioblastoma, renal cancer, lymphoma and leukaemia.

The treatment of cancer also refers to treatment of an established primary tumour or tumours and developing primary tumour or tumours. In one aspect of the invention the treatment of cancer relates to the treatment of metastases. In another aspect of the invention the treatment of cancer relates to treatment of an established primary tumour or tumours or developing primary tumour or tumours.

Therefore according to the present invention, there is provided a method of treating cancer, in a warm-blooded animal, such as man, in need of such treatment which comprises administering to said animal an effective amount of a CHK inhibitor, or a pharmaceutically acceptable salt thereof in combination with an effective amount of a PARP inhibitor, or a pharmaceutically acceptable salt thereof.

According to an aspect of the invention there is provided a pharmaceutical composition that include a CHK inhibitor, or a pharmaceutically acceptable salt thereof, in combination with a pharmaceutical composition which comprises a PARP inhibitor, or a pharmaceutically acceptable salt thereof for use in the treatment of cancer.

According to another feature of the invention there is provided the use of a CHK inhibitor, or a pharmaceutically acceptable salt thereof, in combination with a PARP inhibitor, or a pharmaceutically acceptable salt thereof, in the manufacture of a medicament for use in the treatment of cancer, in a warm-blooded animal, such as man.

Another aspect of the invention provides for the use of a CHK inhibitor, or a pharmaceutically acceptable salt thereof, in combination with a PARP inhibitor, or a pharmaceutically acceptable salt thereof, in the preparation of a medicament for use as an adjunct in cancer therapy or for potentiating tumour cells for treatment with ionizing radiation or chemotherapeutic agents.

Other further aspects of the invention provide inhibiting PARP or CHK activity, comprising administering to a subject a CHK inhibitor, or a pharmaceutically acceptable salt thereof, in combination with a PARP inhibitor, or a pharmaceutically acceptable salt thereof.

Other further aspects of the invention provide inhibiting cell proliferation, comprising administering to a subject a CHK inhibitor, or a pharmaceutically acceptable salt thereof, in combination with a PARP inhibitor, or a pharmaceutically acceptable salt thereof.

In further aspects of the present invention, the CHK/PARP combination may be used in the preparation of a medicament for the treatment of cancer that is deficient in Homologous Recombination (HR) dependent DNA DSB repair activity, or in the treatment of a patient of a cancer which is deficient in HR dependent DNA DSB repair activity.

In another embodiment, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). In one embodiment, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. D., Cancer Treat Res, 107, 29-59 (2002); Janatova M., et al., Neoplasma, 50(4), 246-50 (2003); Jancarkova, N., Ceska Gynekol., 68(1), 11-6 (2003)). Determination of amplification of the BRCA2 binding factor EMSY is described in Hughes-Davies, et al., Cell, 115, 523-535).

The pharmaceutical compositions may be in a form suitable for oral administration, for example as a tablet or capsule, for parenteral injection (including intravenous, subcutaneous, intramuscular, intravascular or infusion), as a sterile solution, suspension or emulsion, for topical administration as an ointment or cream or for rectal administration as a suppository.

Preferably the combination is administered separately, one after another.

In one embodiment, the PARP inhibitor is administered orally and the CHK inhibitor is administered intravenously. In another embodiment, the PARP inhibitor and the CHK inhibitor are both administered orally.

The CHK inhibitor, or a pharmaceutically acceptable salt thereof, will normally be administered to a warm-blooded animal at a unit dose of 1 g or less daily but more than 2.5 mg and this would be expected to provide a therapeutically-effective dose. However the daily dose will necessarily be varied depending upon the host treated, the particular route of administration, and the severity of the illness being treated. Accordingly the optimum dosage may be determined by the practitioner who is treating any particular patient. Particularly the CHK inhibitor could be administered to a warm-blooded animal, at a unit dose of less than 250 mg per day. In another aspect of the invention, the CHK inhibitor could be administered to a warm-blooded animal, at a unit dose of less than 130 mg per day. In a further aspect of the invention, the CHK inhibitor could be administered to a warm-blooded animal, at a unit dose of less than 50 mg per day.

The PARP inhibitor, or pharmaceutically acceptable salt thereof, will normally be administered to a warm-blooded animal at a unit dose, for example, from about 20 mg to 1 g of active ingredient. The PARP can be formulated in a conventional tablet for oral administration containing 50 mg, 100 mg, 250 mg or 500 mg of active ingredient. Conveniently the daily oral dose is above 150 mg, for example, in the range 150 to 750 mg, preferably in the range 200 to 500 mg. For a single dosage form, the active ingredients may be compounded with an appropriate and convenient amount of excipients which may vary from about 5 to about 98 percent by weight of the total composition. Dosage unit forms will generally contain about 20 mg to about 500 mg of each active ingredient. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a kit comprising a CHK inhibitor, as described above, or a pharmaceutically acceptable salt thereof, and a PARP inhibitor as described above, or a pharmaceutically acceptable salt thereof; optionally with instructions for use; for use in the treatment of cancer.

According to a further aspect of the present invention there is provided a kit comprising:
a) a CHK inhibitor, or a pharmaceutically acceptable salt thereof, in a first unit dosage form;
b) a PARP inhibitor, or a pharmaceutically acceptable salt thereof; in a second unit dosage form; and
c) container means for containing said first and second dosage forms; and optionally
d) with instructions for use; for use in the treatment of cancer.

### Examples

### CHK-PARP Combination

Combination experiments were carried out to assess the ability of a CHK inhibitor, i.e., 5-(3-Fluoro-phenyl)-3-ureido-thiophene-2-carboxylic acid (S)-piperidin-3-ylamide, to sensitize cells to a PARP inhibitor, i.e., 4-[3-(4-Cyclopropanecarbonyl-piperazine-1-carbonyl)-4-fluoro-benzyl]-2H-phthalazin-1-one, using a cell viability endpoint.

The cell lines used in this study were SW620, which endogenously express mutant p53, and NCI-H460dominant negative p53 (NCI-H460dnp53), which are stably transfected to express dominant negative p53. Cells were seeded in 96-well plates on day 0 and treated with either a single drug or simultaneously with both drugs for 4 days beginning on Day 1.

For sequential addition, either the CHK inhibitor i.e., 5-(3-Fluoro-phenyl)-3-ureido-thiophene-2-carboxylic acid (S)-piperidin-3-ylamide, or the PARP inhibitor, i.e., 4-[3-(4-Cyclopropanecarbonyl-piperazine-1-carbonyl)-4-fluoro-benzyl]-2H-phthalazin-1-one, were added to cells for 24 hours beginning on Day 1, followed by addition of the other drug on Day 2, and exposure to both drugs continued for an additional 72 hours. The CHK inhibitor was used at a constant concentration at which it had been determined previously to have little or no activity as a single agent. Cells were dosed with the PARP inhibitor to generate a full dose-response curve. See Table 1 for an example of the drug concentrations used in each cell line.

TABLE 1
Compound Concentrations Used (uM)

| | | Top concentration |
|---|---|---|
| Cell Line | CHK Inhibitor | used, PARP inhibitor |
| SW620 | 0.28 | 30 |
| NCI-H460dnp53 | 0.33 | 30 |

Cell viability was measured on day 5 using an MTS colorimetric assay. Dose (concentration of drug used, uM) vs. Fraction unaffected (Fu) for PARP alone versus in combination were plotted. Replicate points for the constant concentration of the CHK inhibitor were also plotted. Data were analyzed by comparing the dose-response curve of the single agent to that for the combination.

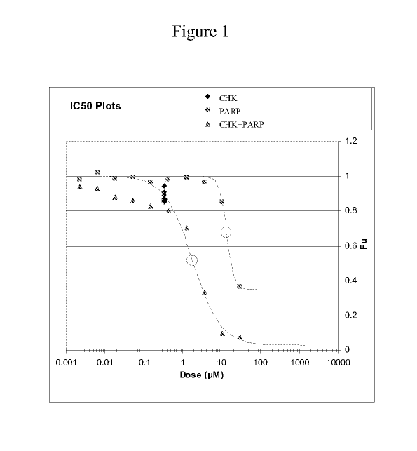

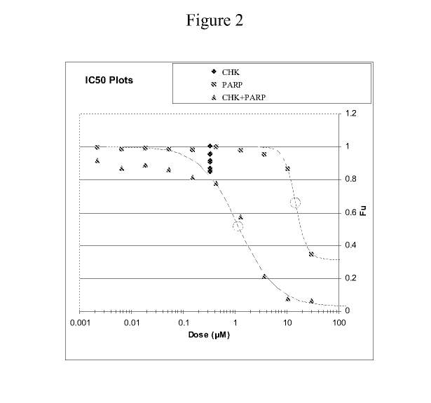

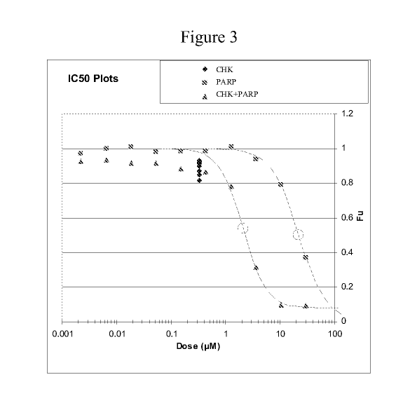

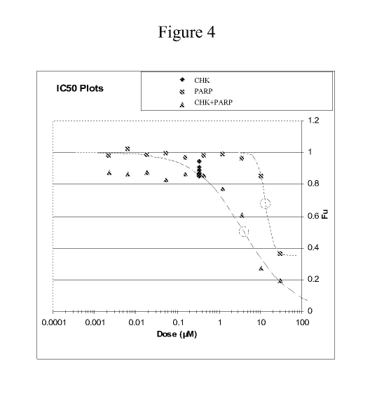

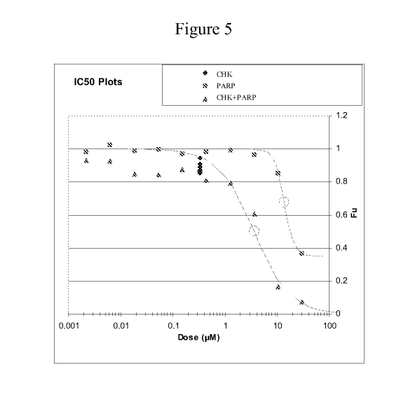

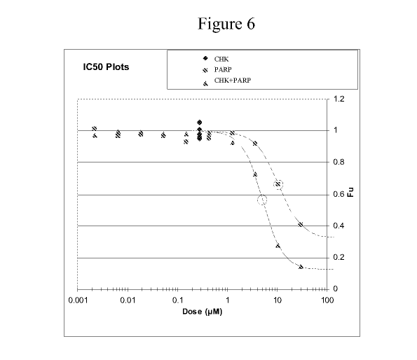

The results showed that cells are relatively insensitive to either compound when used as a single agent. Two cell lines with inactive p53, which have previously been determined to be more sensitive to CHK inhibition than cells expressing wild type p53, were used. The effect of simultaneous compound addition and exposure versus sequential addition of compounds followed by simultaneous exposure was examined (see Table 2 and FIGS. 1-6).

TABLE 2
Representative IC50s* of PARP inhibitor (uM) used as a
single agent vs. in combination with a CHK inhibitor

| Cell Line or | | CHK inhibitor + | CHK inhibitor | PARP inhibitor |
|---|---|---|---|---|
| Number of | PARP inhibitor | PARP inhibitor | followed by | followed by CHK |
| Experiments | alone | Simultaneous | PARP inhibitor | inhibitor |
| NCI-H460dnp53 | 17.6 | 1.9 | 4.1 | 3.6 |
| Number of | 3 | 3 | 1 | 1 |
| experiments | | | | |
| SW620 | 19.4 | 5.8** | No data | No data |
| Number of | 3 | 2 | 1 | 0 |
| experiments | | | | |

*IC = Inhibitory Concentration
**Results are from 2 independent experiments so values are not directly comparable to each other

In vivo studies:

SW 620 tumors were staged in female nude mice as described in Chapter 31 “In vivo Tumor response End Points” by B. A. Teicher (see “Tumor Models in Cancer Research” edited by Beverly A. Teicher, p596. Published by Humana Press Inc. 2002). Treatment started when tumors reached ˜170 mm³. There were 6 groups in this study with n=10/group. PARP inhibitor 4-[3-(4-Cyclopropanecarbonyl-piperazine-1-carbonyl)-4-fluoro-benzyl]-2H-phthalazin-1-one was given 5 times weekly at 50 mg/kg PO. CHK inhibitor 5-(3-Fluoro-phenyl)-3-ureido-thiophene-2-carboxylic acid (S)-piperidin-3-ylamide was given at 12.5 mg/kg and 25 mg/kg twice weekly IV. The combination groups received PARP inhibitor at 50 mg/kg (5 times a week) and, 2 hours later, the CHK inhibitor at 12.5 or 25 mg/kg (twice a week). Mice received 3 weeks of treatment. Overall, treatment was well tolerated except in one case where a mouse was receiving a CHK inhibitor in the 25 mg/kg group it was found dead prior to study endpoint. Results showing the max weight loss % are shown in Table 3 and the efficacy results are shown in Table 4.

TABLE 3

| Inhibitor | Weight loss |
|---|---|
| Vehicle (control) | 1% |
| CHK inhibitor @ 12.5 mg/kg | 2% |
| CHK inhibitor @ 25 mg/kg | 1% |
| PARP inhibitor @ 50 mg/kg | 3% |
| CHK inhibitor @ 12.5 mg/kg + | n.a. |
| PARP inhibitor @ 50 mg/kg | |
| CHK inhibitor @ 25 mg/kg + | n.a. |
| PARP inhibitor @ 50 mg/kg | |

TABLE 4

| Inhibitor | Tumor Growth | |
|---|---|---|
| Vehicle (control) | Inhibition % | |
| AZD 7762 @ 12.5 mg/kg | 15% | p > 0.05 |
| AZD 7762 @ 25 mg/kg | 22% | p > 0.05 |
| AZD 2281 @ 50 mg/kg | 22% | p > 0.1 |
| CHK inhibitor @ 12.5 mg/kg + | 60% | p < 0.001 |
| PARP inhibitor @ 50 mg/kg | | |
| CHK inhibitor @ 25 mg/kg + | 64% | p < 0.001 |
| PARP inhibitor @ 50 mg/kg | | |

The results suggest statistically significant tumor growth inhibition in the combination groups receiving a CHK inhibitor and a PARP inhibitor. No statistically significant activity is observed with either agent alone.

1. A combination comprising a checkpoint kinase (CHK) inhibitor, or a pharmaceutically acceptable salt thereof, and a PARP inhibitor, or a pharmaceutically acceptable salt thereof.

2. A combination according to claim 1 wherein the checkpoint kinase (CHK) inhibitor is selected from a compound of of formula (Ia): 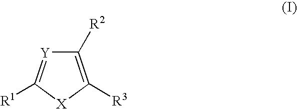
wherein:
X is selected from NH, S and O;
Y is selected from CH or N;
R¹ is selected from cyano, isocyano, C₁₋₆alkyl, —NK¹¹R¹², C₁₋₆alkoxy, C₂₋₆alkenyl, C₂₋₆alkynyl, cycloalkyl, cycloalkenyl, aryl, and heterocyclyl, provided R¹ is not thienyl; and wherein R¹ may be optionally substituted on one or more carbon atoms by one or more R⁹; and wherein if said R¹ contains an —NH— moiety, the nitrogen of said moiety may be optionally substituted by a group selected from R¹⁰;
R² and R³ are each independently selected from —C(═O)NR⁶R⁷, —SO₂NR¹⁶R¹⁷, —NHC(═O)NHR⁴, and —NHC(═NR⁸)NH₂;
R⁴ is selected from H, OH, —NR¹¹R¹², benzyl, C₁₋₆alkoxy, cycloalkyl, cylcoalkenyl, aryl, heterocyclyl, mercapto, CHO, —COaryl, —CO(C₁₋₆alkyl), —CONR³⁰^{R}³¹, —CO₂(C₁₋₆alkyl), —CO₂aryl , —CO₂NR³⁰R³¹, —Salkyl, —SO(C₁₋₆alkyl), —SO₂(C₁₋₆alkyl), —Saryl, —SOaryl, —SO₂aryl, —SO₂NR³⁰R³¹, and —(C₁₋₆alkyl)SO₂NR³⁰R³¹ wherein R⁴ may be optionally substituted on one or more carbon atoms by one or more R¹⁵; and wherein if said heterocyclyl contains a —NH— moiety, the nitrogen may be optionally substituted by a group selected from R¹⁴;
R⁶ and R⁷ are each independently selected from H, OH, OCH₃, C₁₋₆alkoxy, —NH₂, —NHCH₃, —N(CH₃)₂, (C₁₋₃alkyl)NR¹¹R¹², —CH₂CH₂OH, cycloalkyl, and a 5, 6, or 7- membered heterocyclyl ring containing at least one nitrogen atom, provided R⁶ and R⁷ are not both H; alternatively R⁶ and R⁷ taken together with the N to which they are attached form a heterocyclic ring; wherein R⁶ and R⁷ independently of each other may be optionally substituted on one or more carbon atoms by one or more R¹⁸; and wherein if said heterocyclyl contains a —NH— moiety, the nitrogen of said moiety may be optionally substituted by a group selected from R¹⁹;
R⁸ is selected from cyano, isocyano, —SO₂(C₁₋₆alkyl), —SO₂-aryl; —SO₂cycloalkyl, —SO₂cycloalkenyl, —SO₂heterocyclyl, and CF₃; wherein R⁸ may be optionally substituted on one or more carbon atoms by one or more R²³;
R⁹, R¹⁵, R¹⁸, R²³, R²⁴ and R³³ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R⁹, R¹⁵, R¹⁸, R²³, R²⁴ and R³³ independently of each other may be optionally substituted on carbon by one or more R²⁰ and on nitrogen of any moiety that contains an NH or NH₂ by R²¹;
R¹⁰, R¹⁴, R¹⁹, R²⁵ and R³⁴ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R¹⁰, R¹⁴, R¹⁹, R²⁵ and R³⁴ independently of each other may be optionally substituted on carbon by one or more R²² and on nitrogen of any moiety that contains an NH or NH₂ by R²³;
R¹¹ and R¹² are independently selected from H, C₁₋₆alkyl, cycloalkyl, aryl, heterocyclyl; alternatively R¹¹and R¹² taken together with the N to which they are attached form a heterocyclic ring; wherein R¹¹ and R¹² independently of each other may be optionally substituted on carbon by one or more R³³, and wherein if said heterocyclyl contains a —NH— moiety, the nitrogen of said moiety may be optionally substituted by a group selected from R³⁴;
R¹⁶ and R¹⁷ are each independently selected from H, OH, OCH₃, C₁₋₆alkoxy, NH₂, —NHCH₃, —N(CH₃)₂, (C₁₋₃alkyl)NR¹¹R¹², —CH₂CH₂OH, cycloalkyl, aryl, or a 5, 6 or 7-membered heterocyclyl ring containing at least one nitrogen atom, provided R¹⁶ and R¹⁷ are not both H; alternatively R¹⁶ and R¹⁷ taken together with the N to which they are attached form an optionally substituted heterocyclic ring; wherein R¹⁶ and R¹⁷ independently of each other may be optionally substituted on one or more carbon atoms by one or more R²⁴; and wherein if said heterocyclyl contains an —NH— moiety, the nitrogen of said moiety may be optionally substituted by a group selected from R²⁵;
R²⁰, R²² and R³² are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R²⁰, R²¹ and R³²independently of each other may be optionally substituted on carbon by one or more R²⁶ and on nitrogen of any moiety that contains an NH or NH₂ by R²⁷;
R²¹, R²³ and R³⁵ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R²¹, R²³ and R³⁵ independently of each other may be optionally substituted on carbon by one or more R²⁸ and on nitrogen of any moiety that contains an NH by R²⁹;
R²⁶ and R²⁸ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R²⁷ and R²⁹ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰12³¹, —CO(C₁₋₆alkyl), —COheterocyclyl, —COcycloalkyl, —CO₂H, —CO₂(C₁₋₆alkyl), —CO₂(aryl), —CO₂(NR³⁰12³¹), mercapto, —S(C₁₋₆alkyl), —SO(C₁₋₆alkyl), —SO₂(C₁₋₆alkyl), —SO₂NR³⁰12³¹;
R³⁰ and R³¹ are each independently selected from halogen, nitro, —NH₂, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR¹¹R¹², —N(C₁₋₆alkyl)CONR¹¹R¹², —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR¹¹R¹²; wherein R³⁰ and R³¹ independently of each other may be optionally substituted on carbon by one or more R³²; and wherein if said heterocyclyl contains a —NH— or NH₂ moiety, the nitrogen of said moiety may be optionally substituted by a group selected from R³⁵;
or a pharmaceutically acceptable salt thereof;
provided that when X is S; Y is CH; R₂ is C(═O)NR⁶R⁷; and R³ is NHC(═O)NHR⁴; then R¹ cannot be 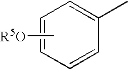
wherein R⁵ is selected from H, optionally substituted carbocyclyl, or optionally substituted C₁₋₆alkyl; with the further proviso that said compound is not
5-Methyl-2-ureido-thiophene-3-carboxylic acid (1-ethyl-piperidin-3-yl)-amide;
[3-((S)-3-Amino-azepane-1-carbonyl)-5-ethyl-thiophen-2-yl]-urea;
2-Morpholin-4-yl-4-ureido-thiazole-5-carboxylic acid (S)-piperidin-3-ylamide;
2-Methyl-5-ureido-oxazole-4-carboxylic acid (S)-piperidin-3-ylamide;
5-(4-CHloro-phenyl)-3-{3-[(R)-1-(2,2,2-trifluoro-acetyl)-piperidin-3-yl]-ureido}-thiophene-2-carboxylic acid (S)-piperidin-3-ylamide; or
N-(3-{[(3S)-3-aminoazepan-1-yl]carbonyl}-5-pyridin-2-yl-2-thienyl)urea;
or a pharmaceutically acceptable salt thereof:

3. A combination according to claim 1 or 2 wherein the PARP is selected from a compound of formula (Ib) 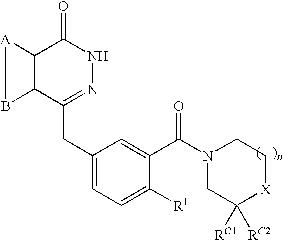
A and B together represent an optionally substituted, fused aromatic ring;
X can be NR^{X} or CR^{X}R^{Y};
if X═NR^{X} then n is 1 or 2 and if X═CR^{X}R^{Y} then n is 1;
R^{X} is selected from the group consisting of H, optionally substituted C₁₋₂₀ alkyl, C₅₋₂₀ aryl, C₃₋₂₀ heterocyclyl, amido, thioamido, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are both hydrogen, or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo,
or a pharmaceutically acceptable salt thereof.

4. A pharmaceutical composition comprising a combination according to claim 1, in association with a pharmaceutically acceptable diluent or carrier.

5. A method of treating cancer₇ in a warm-blooded animal, in need of such treatment which comprises administering to said animal an effective amount of a combination according to claim 1.

6.-8. (canceled)

9. The method according to claim 5 wherein the cancer is oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, brain cancer, renal cancer, lymphoma and leukaemia.

10. The method according to claim 9 wherein the cancer is in a metastatic state.

11. The method according to claim 9 wherein the cancer is in a non-metastatic state.

12. The method according to claim 9 wherein the cancer is renal, thyroid, lung, breast or prostate cancer that is producing bone metastases.
